(19) United States
(12) Patent Application Publication
(10) Pub. No.: US 2008/0085541 A1
(43) Pub. Date: Apr. 10, 2008

(54) Rapid Reverse Transcription of RNA

(75) Inventor: Frank L. Spangler, St. George, UT (US)

Correspondence Address: HOLME ROBERTS & OWEN, LLP, 299 SOUTH MAIN, SUITE 1800, SALT LAKE CITY, UT, 84111, US

(73) Assignee: ADVANCED MOLECULAR SYSTEMS, LLC, St. George, UT (US)

(21) Appl. No.: 11/733,035

(22) Filed: Apr. 9, 2007

Related U.S. Application Data
(60) Provisional application No. 60/828,915, filed on Oct. 10, 2006.

(51) Int. Cl. C12P 19/34

(52) U.S. Cl. 435/91.2; 435/91.1

(57) ABSTRACT

Methods and kits for use in the practice of reverse transcriptase and for one-tube practice of reverse transcriptase and polymerase chain reaction are described. Use of an RNA template having a known conserved area of at least about 40 base pairs, a reverse transcription primer suitable for preparing DNA from the conserved area of said RNA template, and a reverse transcriptase having an elongation rate of at least 40 base pairs per second and a processivity of at least 75 base pairs allows for rapid reverse transcription of the conserved area. Use of PCR primers having 3′ ends which are separated by about 1 to about 120 base pairs, DNA polymerase having an elongation rate of at least 60 base pairs per second and a processivity of at least 150 base pairs together with PCR reaction components allows for rapid amplification of DNA corresponding to the conserved area of the RNA.

### CROSS-REFERENCE TO RELATED APPLICATIONS

This application claims priority to Provisional Application Number 60/828915, entitled “Rapid Reverse Transcriptase for Rapid PCR,” filed on Oct. 10, 2006, which is incorporated herein by reference.

### BACKGROUND OF THE INVENTION

1. The Field of the Invention

The present invention is directed to the field of using RNA as a template to make DNA, and is particularly directed to amplification of resultant DNA.

2. The Relevant Technology

Some procedures, such as the polymerase chain reaction, require the use of DNA or cDNA. Yet, some biological samples contain RNA that is of interest. In such instances, a number of methods exist for making cDNA from an RNA template.

The most common method for making DNA from an RNA sample utilizes a an enzyme called reverse transcriptase, also sometimes called RNA-dependent DNA polymerase. Various reverse transcriptase enzymes are in common use and are supplied commercially.

One conventional method for generating cDNA using reverse transcriptase includes mixing template RNA, primer (sequence specific, poly-T or random hexamer), dNTPs, buffers, reverse transcriptase, and an additional enzyme called RNase inhibitor, which acts to reduce or prevent the degradation of the template RNA. The temperature of this mixture is raised to a desired temperature, typically about 25° C. to about 60° C., depending on the particular reverse transcriptase being used, and transcription is allowed to proceed for about 30 minutes. The result is cDNA that corresponds to the template RNA.

### BRIEF SUMMARY OF THE INVENTION

The present invention provides improvements in the field of reverse transcription of RNA for use in applications such as diagnostics.

Use of an RNA template having a known conserved area of between about 40 and 250 base pairs, a reverse transcription primer suitable for preparing DNA from the conserved area of said RNA template, and a reverse transcriptase having an elongation rate of at least 20 base pairs per second and a processivity of at least 75 base pairs allows for rapid reverse transcription of the conserved area.

The foregoing may be utilized separately, or may accomplished in a single reaction vial along with DNA amplification by combining the RNA template, reverse transcription primer and reverse transcriptase with PCR primers having 3′ ends which are separated by about 1 to about 120 base pairs, DNA polymerase having an elongation rate of at least 60 base pairs per second and a processivity of at least 150 base pairs and the additional PCR reaction components, resulting in rapid amplification of DNA corresponding to the conserved area of the RNA.

These and other objects and features of the present invention will become more fully apparent from the following description and appended claims, or may be learned by the practice of the invention as set forth hereinafter.

### DETAILED DESCRIPTION OF THE PREFERRED EMBODIMENTS

“Point-of-care” (POC) diagnostics are those that are accomplished by a medical service provider or by a patient rather than being sent to a laboratory. In order for a diagnostic test to qualify as POC, it is preferred that the total elapsed time from sample collection to diagnostic results must be completed within 30 minutes. Inasmuch as conventional reverse transcriptase techniques require about 30 minutes just to obtain DNA from an RNA sample, conventional RT is not useful for point-of-care applications since the use of conventional RT leaves no remaining time for amplification of the DNA and for a diagnostic assay to be performed within the overall 30 minute window.

The present invention provides reverse transcriptase (RT) methods and systems which are much faster than conventional RT systems. Rapid reverse transcriptase methods and apparatus are useful in many applications, and are particularly important in applications where time is critical, such as point-of-care diagnostics. A rapid RT procedure utilizing the teachings of the present invention can easily be accomplished in less than 5 minutes, and with care in designing or obtaining optimal materials, in less than about 2 minutes. When coupled with a rapid PCR procedure, it is possible to perform POC diagnostics of RNA within the 30 minute window.

A fragment of DNA having from about 40 to about 250 base pairs in length has been found capable of providing accurate diagnostic results if that DNA fragment is taken from a conserved area of DNA or RNA taken from a biological sample of interest. Hence, in the context of the diagnosis of a sample of RNA, one need not generate a full-length cDNA from the full length of the sample RNA sample, which typically average about 2,500 base pairs (bp) in length. Rather, one can prepare a fragment of DNA corresponding to a conserved area of the sample RNA, which shall often be referred to herein as “template RNA.” A template RNA should have a conserved area of at least about 40 base pairs so that resultant DNA can be accurately diagnosed, and when it is desired to obtain rapid reverse transcription and/or rapid subsequent PCR, this is preferred that the conserved area of the RNA template be no greater than about 250 base pairs.

Although those of ordinary skill will appreciate applications for rapid RT outside its use in connection with POC applications, for purposes of brevity, the following description shall focus on an exemplary one-tube method involving rapid reverse transcription followed by amplification using the polymerase chain reaction (PCR).

In order to avoid degradation of template RNA over time, which could lead to erroneous results, it is preferred that a sample containing the RNA template be subjected to rapid RT within five minutes from extraction, and it is more preferred that it be accomplished within one minute from extraction.

Although it has been observed that reverse transcription occurs when RNase inhibitors have been added in the conventional fashion, it has been discovered that reverse transcription is even faster in the absence of RNase inhibitors. Hence, it is preferred that no RNase inhibitors be included in the reverse transcription reaction mixture.

For a POC application, the RNA segment of interest should preferably have a known conserved area of between about 40 and about 250 base pairs in length, although as noted above, larger conserved areas are still capable of being utilized if the amount of increased time required for reverse transcription is acceptable.

For POC applications, primer and probe sequences should be obtained through selection or preparation which, in concert with optimization of reagents in the reaction mixture, allows the reverse transcription step to be completed in less than five minutes, and more preferably in less than two minutes, resulting in formation of a DNA fragment that corresponds to the known conserved area of the template RNA.

DNA primers used in the amplification step should be obtained through selection or preparation that have 3′ ends separated by about 1 to about 120 base pairs. Inasmuch as primers are typically about 20 base pairs long, this results in an amplicon that is in the range of about 40 to about 160 base pairs in length.

Elongation rates of different specific RT and polymerase enzymes range from about 23 bp/second at the slow end to about 200 bp/second at the fast end. A fast elongation rate allows the RT-PCR reaction to be completed more rapidly, so in the context of a POC application, it is desired to have a rapid elongation rate. It has been found that an elongation rate of about 20 bp/second or greater is typically acceptable, although it is an aspect of the invention to obtain RT and polymerase enzymes through selection or preparation that have an elongation rate of greater than about 50 bp/second for POC applications.

The “processivity” of RT and polymerase enzymes is also important when desiring a fast reaction, such as in a POC application. “Processivity” is a measure of the average number of nucleotides added by a DNA polymerase enzyme per association/disassociation with the template. If the enzyme falls off of the template too soon, it must reattach to form a DNA fragment of useful length. In the context of an RT-PCR POC application, it is preferred that the polymerase enzyme and the RT enzyme have an average processivity greater than about 75 bp, which will usually result in a complete copy from the conserved area of the RNA in one or two passes, i.e., the entire amplicon is generated off of one or two elongation events. It is preferred that the RT enzyme have an average processivity of greater than about 150 bp since that will substantially increase the number amplicons being generated off of a single elongation event. There is no upper limit to the length of the RT enzyme since the length of the amplicon controls the upper limit of the replication step.

The polymerase primers are obtained through selection or design so as to have useful melting temperatures (Tm), preferably but not necessarily between about 50 and 60 degrees C. It is preferred that both primers be selected or designed so as to have a similar melting temperatures so as to avoid the problem of allowing one of the primers to attach and be replicated significantly prior to the other.

In tests, all of the constituents of the RT-PCR mixture that are consumed were provided in excess concentration so as to increase nucleation events with the RNA template material. Following is a range of concentrations that have been demonstrated to be useful in the practice of the RT-PCR process:

0.9 to 1.5 pmol per microliter of both forward and reverse PCR primers

0.1 to 0.2 pmol per microliter of a fluorogenic probe

9.0-11.0 mM Tris pH 8.3

1.0-3.0 mM MgCl₂

0.2-0.5 mM dNTP (each)

40-60 mM KCl

0.04-0.2 Units per microliter of thermostable DNA polymerase

0.2-0.4 Units per microliter of reverse transcriptase

0.08-100.0 ng of the template RNA

Dissolved/Suspended in Molecular Grade Water

The foregoing mixture was introduced into a single sample vial and placed into a “rapid” PCR thermocycler, by which it is meant that the thermocycler was capable of temperature transition rates of at least about 5.0 degrees C. per second. However, it should be understood that advantages of the invention can be obtained in thermocyclers having a lower temperature transition rate.

A thermocycler device of the type discussed in copending application Ser. No. 11/697,917 entitled “Rapid Thermocycler” filed Apr. 9, 2007, incorporated herein by reference, was utilized to perform RT-PCR tests according to this example.

It is preferred that a rapid thermocycler be operated according to the following protocol, again bearing in mind that the rapid thermocycler utilized in the tests was capable of temperature transition rates of at least about 5.0 degrees C. per second, and that other thermocyclers would require alterations to the method:
Bring the sample to a suitable temperature, e.g. 48 degrees C., and allow the RT reaction to proceed (about 2 minutes to about 5 minutes, depending upon materials) so as to prepare template DNA;
Bring the sample to a suitable temperature, e.g. about 95 degrees C., for a sufficient time, e.g. about 20 seconds to 10 minutes, in order to dissociate the template DNA, actuate the polymerase, and denature the reverse transcriptase;
Repeat the following two temperature-holds approximately 40 times so as to amplify the DNA until the amount of DNA is sufficient for the diagnostic test:
   95° C., or other suitable dissociation target temperature for an appropriate time, e.g. for 1 second; and
   60° C., or other suitable annealing/elongation target temperature for an appropriate time, e.g. for 10 seconds.

The amplified DNA of the foregoing method was then subjected to analysis, which can be done in any convenient manner, including real-time analysis, gel electrophoresis or other suitable method. POC applications, of course, require rapid analysis capable of providing the type of information required for a POC determination, which can range from a simple determination of the presence or absence of particular genetic materials, to something more detailed or quantitative.

It will be appreciated that specific details set forth in the foregoing description have been given in order to provide a thorough understanding of the present invention, but it will be apparent to one skilled in the art that the present invention may be practiced without these specific details or with different details. In many respects, well-known aspects of reverse transcription and PCR processes have not been described in particular detail in order to avoid unnecessarily obscuring the present invention.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for obtaining DNA from an RNA template, comprising the steps of:
obtaining an RNA template having a known conserved area of at least about 40 base pairs;
obtaining a primer suitable for preparing DNA from the conserved area of said RNA template;
obtaining a reverse transcriptase having an elongation rate of at least 20 base pairs per second and a processivity of at least 75 base pairs;
providing a reaction mixture containing constituents necessary for reverse transcription along with said RNA template, primer and reverse transcriptase; and
heating said reaction mixture to a suitable temperature for less than five minutes in order to obtain DNA corresponding to the conserved area of the template RNA.

2. The method of claim 1, wherein the reverse transcriptase has an elongation rate of at least 50 base pairs per second.

3. The method of claim 1, wherein the reverse transcriptase has a processivity of at least 150 base pairs.

4. The method of claim 1, further comprising the step of commencing the step of heating the reaction mixture to a suitable temperature within five minutes of extraction of a sample containing the RNA template.

5. The method of claim 1, further comprising the step of commencing the step of heating the reaction mixture to a suitable temperature within one minute of extraction of a sample containing the RNA template.

6. The method of claim 1, wherein the reaction mixture is free of added RNase inhibitors.

7. A method for obtaining DNA from an RNA template, comprising the steps of:
obtaining an RNA template having a known conserved area of at least about 40 base pairs;
obtaining a primer suitable for preparing DNA from the conserved area of said RNA template;
obtaining a reverse transcriptase having an elongation rate of at least 20 base pairs per second and a processivity of at least 75 base pairs;
obtaining primers suitable for use in amplification of DNA, said primers having 3′ ends which are separated by about 1 to about 120 base pairs;
obtaining DNA polymerase having an elongation rate of at least 60 base pairs per second and a processivity of at least 150 base pairs;
providing a reaction mixture containing constituents necessary for reverse transcription and DNA amplification along with said RNA template, primer, reverse transcriptase, amplification primers and DNA polymerase;
heating said reaction mixture to a suitable temperature in order to obtain DNA corresponding to the conserved area of the template RNA by operation of the reverse transcription enzyme;
heating said reaction mixture to an elevated temperature for a time sufficient to activate the DNA polymerase and to denature the reverse transcriptase enzyme;
bringing said reaction mixture to target temperatures for a period of time suitable for effecting dissociation of DNA, then annealing, then elongation, and repeating the bringing of the reaction mixture to said target temperatures as many times as desired in order to obtain a desired amount of DNA.

8. The method of claim 7, wherein the reverse transcriptase has an elongation rate of at least 50 base pairs per second.

9. The method of claim 7, wherein the reverse transcriptase has a processivity of at least 150 base pairs.

10. The method of claim 7, further comprising the step of commencing the step of heating the reaction mixture to a suitable temperature within five minutes of extraction of a sample containing the RNA template.

11. The method of claim 7, further comprising the step of commencing the step of heating the reaction mixture to a suitable temperature within one minute of extraction of a sample containing the RNA template.

12. The method of claim 7, wherein the reaction mixture is free of added RNase inhibitors.

13. The method of claim 7, wherein the step of heating said reaction mixture to a suitable temperature in order to obtain DNA corresponding to the conserved area of the template RNA by operation of the reverse transcription enzyme is performed in no more than five minutes.

14. The method of claim 7, wherein the step of heating said reaction mixture to a suitable temperature in order to obtain DNA corresponding to the conserved area of the template RNA by operation of the reverse transcription enzyme is performed in no more than about two minutes.

15. The method of claim 7, wherein the temperature transition rates between the target temperatures for effecting dissociation of DNA, then annealing, then elongation, is at least 5.0 degrees C. per second.

16. The method of claim 7, wherein the step of heating said reaction mixture to a dissociation target temperature suitable for effecting dissociation of DNA is concluded in about one second.

17. The method of claim 7, wherein the step of cooling said reaction mixture to an annealing/elongation temperature suitable for effecting annealing and elongation is concluded in about ten seconds.

18. The method of claim 7, wherein the step of bringing said reaction mixture to target temperatures for a period of time suitable for effecting dissociation of DNA, then annealing, then elongation, and repeating the bringing of the reaction mixture to said target temperatures as many times as desired in order to obtain a desired amount of DNA comprises the steps of:
heating said reaction mixture to a dissociation target temperature for a length of time suitable for effecting dissociate of DNA;
cooling said reaction mixture to a temperature suitable for effecting annealing and elongation for a length of time suitable to effect annealing and elongation; and
repeating said heating and cooling of the reaction mixture between the dissociation and annealing/elongation temperatures as many times as desired in order to obtain a desired amount of DNA.

19. A kit for preparation of DNA from an RNA sample, comprising:
a reverse transcription primer suitable for preparing DNA from a target RNA template having a known conserved area of at least about 40 base pairs;
a reverse transcriptase having an elongation rate of at least 20 base pairs per second and a processivity of at least 75 base pairs; and
additional reaction mixture constituents necessary for reverse transcription along with said reverse transcription primer, reverse transcriptase.

20. The kit of claim 19, wherein the additional reaction mixture does not contain RNase inhibitors.

21. A kit for preparation of DNA from an RNA sample, comprising:
a reverse transcription primer suitable for preparing DNA from a target RNA template having a known conserved area of at least about 40 base pairs;
a reverse transcriptase having an elongation rate of at least 20 base pairs per second and a processivity of at least 75 base pairs;
forward and reverse primers suitable for use in amplification of DNA formed by reverse transcription of said target RNA, said primers having 3′ ends which are separated by about 1 to about 120 base pairs;
DNA polymerase having an elongation rate of at least 50 base pairs per second and a processivity of at least 150 base pairs; and
additional reaction mixture constituents necessary for reverse transcription and DNA amplification along with said reverse transcription primer, reverse transcriptase, forward and reverse DNA amplification primers and DNA polymerase.

22. The kit of claim 21, wherein the additional reaction mixture does not contain RNase inhibitors.
